(19) United States
(12) Patent Application Publication
(10) Pub. No.: US 2026/0358484 A1
(43) Pub. Date: Nov. 12, 2026

(54) SYSTEMS AND METHODS FOR TRANSDUCER ELEMENT DETECTION

(71) Applicant: LUMA Vision Limited, Dublin (IE)

(72) Inventors: Javier Francisco Esteban, München (DE); Raghavendra Padmanabha, Munich (DE)

(21) Appl. No.: 19/669,087

(22) Filed: May 6, 2026

Related U.S. Application Data
(60) Provisional application No. 63/801,391, filed on May 7, 2025.

(51) Int. Cl. G01S 7/52; A61B 8/00; G01R 27/16

(52) U.S. Cl. CPC G01S 7/5205; A61B 8/4488; A61B 8/58; G01R 27/16

(57) ABSTRACT

The invention relates to systems and methods transducer element detection and control using a smart analog front end (AFE) utilizing a novel analog mixer, and a signal generator and gain-phase measurement unit, in combination with a standard ultrasound analog front end to detect dead channels, match the output of the pulser to channel impedance, and measure the crosstalk as a calibration step.

### CROSS-REFERENCE TO RELATED APPLICATIONS

This application claims priority to, and the benefit of, U.S. Provisional Application No. 63/801,391, filed May 7, 2025, the content of which is incorporated by reference herein in its entirety.

### FIELD OF THE INVENTION

The invention generally relates to ultrasound imaging, and, more particularly, to systems and methods for transducer probe element detection using a smart analog front end.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image. In particular, received echoes are converted to electrical signals and transmitted to processing equipment, in which a resulting ultrasound image of the area of interest may be displayed. Accordingly, medical ultrasound imaging includes the control and processing of a variety of high-speed signals, including high-frequency sound waves, high-frequency/wide-dynamic-range continuous/pulsed waves, high-speed digital processing and video display of digital images.

The performance of ultrasound systems is determined by, among other factors, the system channel count of the transmitting and receiving signal chain, or ultimately the transmitting and receiving aperture size. For example, catheter-based endovascular ultrasound imaging technologies employed within the vasculature e.g. intravascular ultrasound (IVUS) or intracardiac echocardiography (ICE), typically utilize a multielement transducer assembly attached to a distal end of a catheter. The transducer assembly transmits ultrasound waves and receives echoes from those waves. In typical ultrasound systems configured to visualize inner body regions, multielement transducer probes consisting of 128-512 elements arranged in linear or curvilinear arrays are used to transmit and receive ultrasound signals across an aperture formed by the multiple transducer elements. When visualizing inner body regions, dynamic forces and movements make it difficult to stabilize internal imaging devices and to generate consistent and accurate images if imaging of the structure cannot be enabled in real-time (e.g., >20 Hz).

Because the captured images from IVUS and/or ICE systems often lack the necessary quality required to prescribe appropriate treatment or therapy, current ultrasound technology advancements are directed toward achieving higher resolution images, which can be displayed in 3D or 4D, and can be displayed in real time. Further, current clinical applications demand cost-effective, high-performance systems, including ultra-portable systems for advanced IVUS and ICE imaging, and to bring modern medical imaging technology to remote areas for point-of-care applications. These needs involve reductions in power requirements and in the size of ultrasound components including analog front end and transmitter chip technology.

Further, in order to display higher resolution images in real time, current ultrasound technologies must address the ability to connect to more transducer channels simultaneously, as well as including higher performance processing capabilities to process more frames per second. While more channels derive larger acoustic aperture, or better lateral resolution, and better signal-to-noise ratio, more channels also increase the system size, cost and power requirements. Accordingly, difficult engineering tradeoffs still exist between system size, complexity, cost, and achievable image quality. Thus, there is a need for improved ultrasound imaging systems that meet current clinical needs.

### SUMMARY

The present invention recognizes the limitations of current Analog Front Ends (AFEs) used for ultrasound imaging and provides systems and methods utilizing a smart AFE to address the drawbacks of current systems. In particular, the systems and methods of the invention provide for automatically detecting open AFE channels in multi-element transducers and masks these elements during pulsing to protect the ultrasound channel from damage by the high voltage pulses generated from the system. In turn, this protects the various components of the system, such as the pulser, the receiver, and the electronic components that control the system from the damage caused by the reflection of energy. Accordingly, systems of the invention provide improved reliability of connected channels in ultrasound devices and allow for pulsing at higher voltages.

When the AFE channels are opened, i.e. not connected to an active transducer element, the channel can be damaged if the transducer is not connected during operation. This is because the high voltage pulse generated by the system will not have a load to dissipate into, causing energy to be reflected back into the system. Thus, AFEs may be limited to a certain pulse amplitude when the channels are opened to avoid damage to the various components within the system. As an example, a system with an impedance of 70 ohms may be safely pulsed at 100V but may have an actual capability of pulsing at 200V. However, this voltage could damage the AFE channel if it is not loaded by an active ultrasound element. Not knowing the exact dead ultrasound channels at a given time point prevents the user from pulsing at higher volts. Systems of the invention solve this problem by providing a smart AFE with an integrated fixed impedance measurement to dynamically provide information about dead and active channels and to automatically mask elements between every pulsing cycle.

Aspects of the invention include systems including an analog front-end (AFE) operably couplable to a multi-element transducer of an ultrasound imaging device via a plurality of element channels; and a console operably coupled to the AFE and further configured to be operably associated with the ultrasound imaging device and exchange data therewith, wherein the console comprises a hardware processor coupled to non-transitory, computer-readable memory containing instructions executable by the processor. The instructions executable by the processor cause the AFE to automatically detect one or more dead element channels when the AFE is coupled to the multi-element transducer of an ultrasound imaging device; and mask the detected one or more dead element channels such that, during an ultrasound pulse cycle, application of an ultrasound pulse to the one or more dead element channels is prevented, wherein masking the one or more dead element channels allows for pulsing at higher volts.

In some embodiments, masking is performed on receiving. In this way, dead channels, which contribute to darken the image during beamforming are masked.

In some embodiments the AFE comprises an impedance measurement circuit comprising a signal generator and gain and phase measurement circuit, wherein the impedance measurement circuit detects the one or more dead element channels.

In particular embodiments, the impedance measurement circuit is configured to analyze an element channel against a preset low voltage, fixed frequency signal to determine whether an impedance measurement of an element channel is within a designated impedance limit. Further, the impedance measurement circuit measures the impedance of the plurality of element channels before every pulse, in some embodiments. In some embodiments, the signal generator, in conjunction with the gain and phase measurement circuit, are configured to measure the impedance of the channel whenever a channel is not being used. In some embodiments, the AFE further comprises a field programmable gate array (FPGA), an ultrasound analog circuit, and a high speed analog switch, wherein the impedance measurement is relayed to the FPGA, wherein the FPGA masks the open channel. In some embodiments, the impedance measurement is used as an input to set the output impedance of an ultrasound pulser, wherein matching the output impedance to an element channel impedance improves signal strength.

In some embodiments, the AFE is further configured model a crosstalk using the impedance measurements of two element channels. Further, the modeled crosstalk is used for calibration in one or more of image reconstruction and/or rendering, in some embodiments.

In some embodiments, the AFE comprises a circuit comprising a multiplexer and a measurement device, wherein the multiplexer sequentially connects each of the element channels with the measurement device. For example, the measurement device measures the circuit at one or more points to determine the one or more dead element channels and one or more alive channels, wherein the measurement device produces a binary output of an element channel as dead or alive, in some embodiments. In particular embodiments, the measurement device is an impedance analyzer. Further, in some embodiments, the impedance measurement is measured at a known frequency with a known value. In some embodiments, the element channel is detected as a dead channel if the impedance measurement exceeds a threshold value. Further, when an element channel is detected as dead, the measurement device communicates with the AFE such that AFE prevents application of the ultrasound pulse to the element channel, in some embodiments.

Aspects of the invention provide methods comprising providing a system comprising an analog front-end (AFE) operably couplable to a multi-element transducer of an ultrasound imaging device via a plurality of element channels and a console operably coupled to the AFE and further configured to be operably associated with the ultrasound imaging device and exchange data therewith; detecting one or more dead element channels when the AFE is coupled to the multi-element transducer of an ultrasound imaging device; and masking the detected one or more dead element channels such that, during an ultrasound pulse cycle, application of an ultrasound pulse to the one or more dead element channels is prevented, wherein masking the one or more dead element channels allows for pulsing at higher volts.

In some embodiments, masking is performed on receiving. In this way, dead channels, which contribute to darken the image during beamforming are masked.

In some embodiments of the methods, the AFE comprises an impedance measurement circuit comprising a signal generator and gain and phase measurement circuit, wherein the impedance measurement circuit detects the one or more dead element channels. Further, in some embodiments, the impedance measurement circuit is configured to analyze an element channel against a preset low voltage, fixed frequency signal to determine whether an impedance measurement of an element channel is within a designated impedance limit. Further, the impedance measurement circuit measures the impedance of the plurality of element channels before every pulse, in some embodiments. In other embodiments, the signal generator, in conjunction with the gain and phase measurement circuit, are configured to measure the impedance of the channel whenever a channel is not being used. In some embodiments, the AFE further comprises a field programmable gate array (FPGA), an ultrasound analog circuit, and a high speed analog switch, wherein the impedance measurement is relayed to the FPGA, wherein the FPGA masks the open channel. In some embodiments, the impedance measurement is used as an input to set the output impedance of an ultrasound pulser, wherein matching the output impedance to an element channel impedance improves signal strength.

In some embodiments of the methods, the AFE is further configured model a crosstalk using the impedance measurements of two element channels. Further, the modeled crosstalk is used for calibration in one or more of image reconstruction and/or rendering, in some embodiments.

In some embodiments of the methods, the AFE comprises a circuit comprising a multiplexer and a measurement device, wherein the multiplexer sequentially connects each of the element channels with the measurement device. In particular embodiments, the measurement device measures the circuit at one or more points to determine the one or more dead element channels and one or more alive channels, wherein the measurement device produces a binary output of an element channel as dead or alive.

In some embodiments of the methods, the measurement device is an impedance analyzer. In particular embodiments, the impedance measurement is measured at a known frequency with a known value. Further, the element channel is detected as a dead channel if the impedance measurement exceeds a threshold value, in some embodiments. For example, in some embodiments, when an element channel is detected as dead, the measurement device communicates with the AFE such that AFE prevents application of the ultrasound pulse to the element channel.

### BRIEF DESCRIPTION OF THE DRAWINGS

FIGS. 1A and 1B are diagrammatic illustrations of an ultrasound system with which systems of the invention may be coupled.

FIG. 2 is a perspective view of an imaging catheter with which systems of the invention may be coupled.

FIG. 3 is a schematic of an AFE according to one embodiment of systems of the invention. In the figure below, the signal generator, along with the gain and phase measurement circuit, measures the impedance of the channel whenever a channel is not being used. This information is relayed to the FPGA, which can mask the channels.

FIG. 4 is a schematic illustration of an AFE according to one embodiment of systems of the invention.

FIG. 5 illustrates a block diagram of one embodiment of methods for probe element detection and control using systems of the invention.

### DETAILED DESCRIPTION

The present invention recognizes the limitations of AFEs currently utilized in ultrasound devices, namely the limitation to certain pulse amplitudes due to open channels and the potential damage to the ultrasound components that may result. In particular, the systems and methods of the invention provide for dynamically and automatically detecting and masking open element channels in multi-element transducers during pulsing to protect the AFE channel from damage by the high voltage pulses generated from the system. In turn, this protects the various components of the system, such as the pulser, the receiver, and the electronic components that control the system from the damage caused by the reflection of energy. Accordingly, systems of the invention provide improved reliability of connected channels in ultrasound devices and allow for pulsing at higher voltages, thus delivering superior ultrasound image quality. In some embodiments, masking is performed on receiving. In this way, dead channels, which contribute to darken the image during beamforming are masked thus also improving image quality.

### Overview

The invention provides systems and methods for automated, dynamic transducer probe element detection and control using a smart analog front end (AFE). As is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). The imaging device, (i.e. the transducer, probe, or transducer probe) may be placed inside a body opening (e.g. endovascular ultrasound, intravascular ultrasound, intracardiac echocardiography). The final quality of the image obtained through ultrasound scanning is limited to the technical specifications of the equipment, the propagation of ultrasonic waves through the tissue analyzed, and the method used to reconstruct the images.

Many probes utilize multielement transducers to shape and steer the transmitted ultrasonic waves, and to receive echo signals across an aperture formed by a plurality of the transducer elements. The received echo signals are processed by the ultrasound system which controls the activation of the transducer and processes the received echo information to form an image or measure a physiological condition of the body, for example, the state of flow in vessels of the body. The individual elements are electrically connected to individual channels of a processor known as a beamformer. Each channel of the beamformer controls the time of activation of its respective transducer element and appropriately delays the echo signals received by a transducer element to bring the echo signal components received by the transducers into time coincidence. The time aligned echo signal components can then be combined to form a coherently focused echo signal. The coherent echo signal can then be detected and further processed as a contributor to a spatial image of the interior of the body or to measure flow and other functions of the body.

When the channels are opened, i.e. not connected to an active ultrasound element, the AFE channel can be damaged if the transducer is not connected during operation because the high voltage pulse generated by the system will not have a load to dissipate into, causing energy to be reflected back into the system. AFE channels that are not loaded by an active ultrasound element, i.e. interchangeably referred to herein as channels that are opened, open channels, and, dead channels, may occur during the build of the catheter, and/or due to aging. Not knowing the exact dead channels at a given time prevents pulsing at higher volts because of the risk of damaging AFE channels that are not loaded by an active ultrasound element and/or the other components of the ultrasound system. Thus, AFEs may be limited to a certain pulse amplitude when the channels are opened to avoid damage to the various components within the system. By way of illustration, some systems may have an impedance of 70ohms, with a safe pulse voltage of 100V. While these systems may be able to pulse at 200V, this pulsing voltage could damage the AFE channel if it is not loaded by an active US element. Systems of the invention solve this problem by providing a smart AFE with a fixed impedance measurement integrated into the AFE to provide information on open channels and to dynamically mask the elements between every pulsing cycle.

### Ultrasound imaging system

Systems of the invention may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Patent No. 11,382,599 to Hennersperger et al., the contents of each which are incorporated by reference herein. The data may be processed using imaging protocols to extract anatomical and functional information, and tissue characteristics as disclosed in more detail herein, and as disclosed in International PCT Application No. PCT/IB2019/000963 (Published as WO 2020/044117) to Hennersperger et al., U.S. Application Publication No. US 2022-0287679A1 to Hennersperger et al., and U.S. Patent No. 11,382,599 to Hennersperger et al., the contents of each which are incorporated by reference herein.

The systems may include a console configured to be operably associated with one or more devices such as an ultrasound imaging device and to exchange data therewith. The console may comprise a hardware processor coupled to non-transitory, computer-readable memory containing instructions executable by the processor to cause the console to receive data associated with a complex anatomy. For example, the console may be operable to receive a plurality of data, process and combine the data, and reconstruct, based on the processing and combining of the data, an interactive digital model of an imaged anatomy. In some embodiments, the complex anatomy may be cardiac and/or vascular anatomy. The data may be, for example, both catheter-based ultrasound imaging data and pulse phase data.

The console may be in active communication with a computing system configured to communicate across a network. The computing system or computing device may include one or more processors and memory, as well as an input/output mechanism (i.e., a keyboard, knobs, scroll wheels, or the like) with which a user can interact so as to operate the console, including making adjustments to the ultrasound imaging system, saving images, initialization, continuous 3D image registration, filtering, optimization, 3D fusion, and panoramic image reconstruction.

The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which may be stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

During operation, the CPU and/or GPU may control the transmission and receipt of electrical currents, subsequently controlling the emission and receipt of sound waves from the probe. The CPU and/or GPU may also analyze electrical pulses that the probe makes in response to reflected waves coming back and convert this data into images (i.e., ultrasound images) that can then be viewed on a display, which may be an integrated monitor. Such images may also be stored in memory and/or printed via a printer.

The systems may be configured to receive three-dimensional (3D) ultrasound image data from an imaging device. In some embodiments, the invention provides for reconstruction of a patient-specific anatomical model for use in minimally invasive procedures in the vasculature. Accordingly, ultrafast ultrasound imaging techniques, such as planewave or diverging wave imaging, may be required to enable imaging within the constraints of the application, particularly for intravascular and/or intracardiac tissue assessment and analysis. These constraints could be posed due to the high temporal update rate as required for effects observed in visualization and tissue characterization, where plane and diverging wave methods enable high imaging rates, commonly also referred to ultrafast imaging approaches. Systems and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques. Thus, the 3D ultrasound image data received by systems of the invention may be real-time 3D ultrasound data. For example, the data may be full circumferential, three-dimensional (3D) image data.

While exemplary embodiments describe ultrasound imaging data received from 3D ICE catheters, catheter-based ultrasound imaging as described herein is not limiting. Imaging data may be received from catheter-based ultrasound systems that may include endovascular and/or intravascular ultrasound for in-body applications. This may include systems and imaging data for imaging in large cavities of the heart, coronary and peripheral arteries, as well as other organs such as liver, kidney, and the like. Systems and methods of the invention may be configured to receive information on ablation electrodes, e.g. spline electrodes on basket catheters, or single electrodes on a point by point catheter.

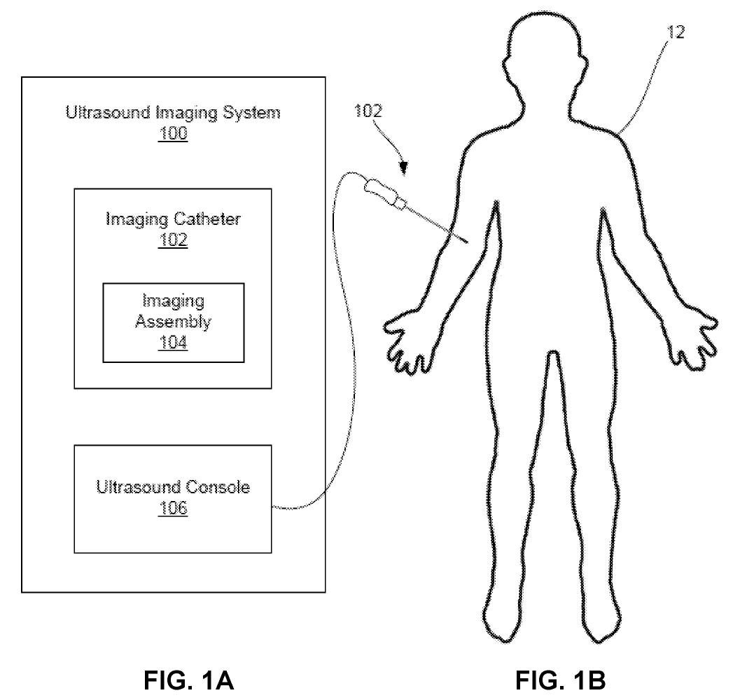

FIGS. 1A and 1B are diagrammatic illustrations of an exemplary ultrasound system 100 for providing catheter-based ultrasound imaging data specific to a patient 12. The system 100 may include an imaging device 101 equipped with an imaging assembly 104 and a console 106 to which the imaging device 101 is to be connected. The imaging device may be an imaging catheter 102. Accordingly, systems and methods for interactive reconstruction of patient-specific digital model of cardiac and/or vascular anatomy may use a 4D Intracardiac echocardiogram (ICE) system that captures the anatomy of interest.

As disclosed in detail herein, the imaging device may generally be in the form of an imaging catheter capable of providing imaging and mapping capabilities. Accordingly, such a device may be useful for ultrasound visualization of intravascular and/or intracardiac tissue, which may be particularly useful for catheter-based interventional procedures for assessing the anatomy as well as functional data in relation to a target volume of interest.

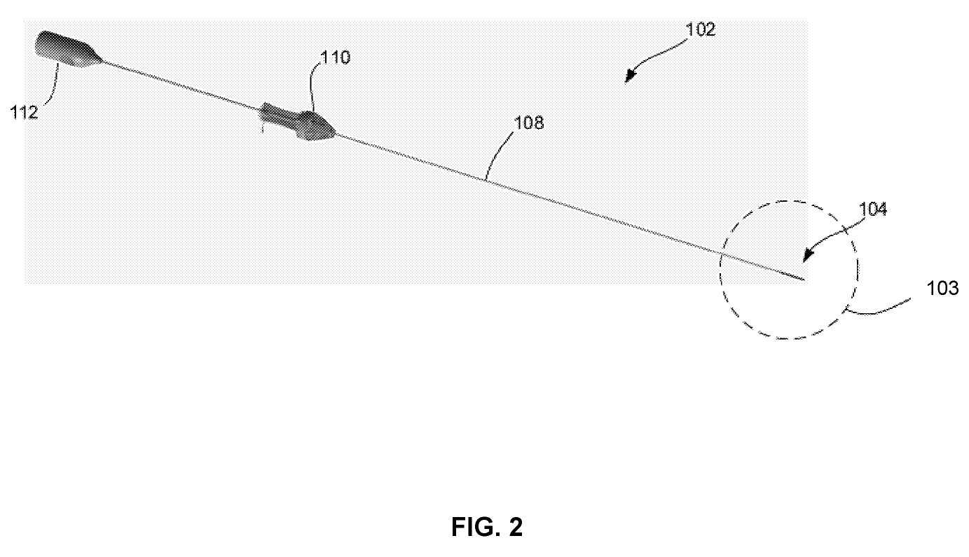

FIG. 2 is a perspective view of an imaging catheter 102 with which systems of the invention may be coupled. The catheter 102 may include a catheter body 108, including proximal and distal portions. The imaging assembly 104 may be provided at the distal portion 103, for example, generally defining a distal end of an imaging catheter. A handle 110 may be operably associated with the catheter body 108 and allow for an operator (i.e., surgeon or other medical professional) to manipulate and advance the imaging assembly 104 and the catheter body 108 to a desired target site within the patient’s vasculature. The handle 110 may include user-operable inputs for controlling various features and functions of the imaging assembly 104. An interface member 112 may be provided at a distal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

As generally understood, the systems and methods of the present invention may be used for ultrasound visualization of tissue of any kind with respect to any kind of procedure in which imaging analysis is used and/or preferred. The imaging device may be useful in carrying out catheter ablation to treat a cardiac condition, such as atrial fibrillation (AF) or the like. For example, in some embodiments, the catheter may include components providing associated capabilities. For example, portions of the catheter may include sensors (e.g., localization and/or tracking sensors) and/or energy delivery elements (e.g., ablation elements).

The imaging catheter may include a fully rotatable transducer unit comprised of an ultrasound transducer array configured to transmit ultrasound pulses to, and receive echoes of the ultrasound pulses from, surrounding intravascular tissue during a procedure. Such ultrasound transmissions result in a collection of image data which is received by the console and subsequently reconstructed into one or more images providing visualization and characterization of the surrounding intravascular tissue. In particular, the console may utilize image data received from an imaging assembly of the imaging catheter to reconstruct one or more images, including at least 2D and 3D images of the anatomical region of interest (i.e., intravascular and/or intracardiac tissue).

The console may process the received image data utilizing certain imaging protocols and algorithms for reconstructing images and subsequently outputting, via a display, the reconstructed images to an operator depicting visualization of the anatomical region of interest.

In addition to providing reconstruction of images based on received image data from the imaging assembly, the console may further provide control over the imaging assembly, including control over the emission of ultrasound pulses therefrom (intensity, frequency, duration, etc.), control over the movement of the ultrasound transducer unit (i.e., controlling rotation, including speed and duration of rotation), and control over masking the dead element channels.

### Systems for probe element detection and control using a smart AFE

As noted above, medical ultrasound requires the control and processing of a variety of high-speed signals. Those signals include high-frequency sound waves, high-frequency/wide-dynamic-range continuous/pulsed waves, high-speed digital processing and video displays. Systems of the invention provide both continuous and discrete solutions for probe element detection and control using a smart AFE. As discussed in more detail herein, the distinctive features of the smart AFE systems of the invention include a novel analog mixer, and a signal generator and gain-phase measurement unit, in combination with a standard ultrasound analog front end. This enables the AFE to detect dead channels, match the output of the pulser to channel impedance, and measure the crosstalk as a calibration step.

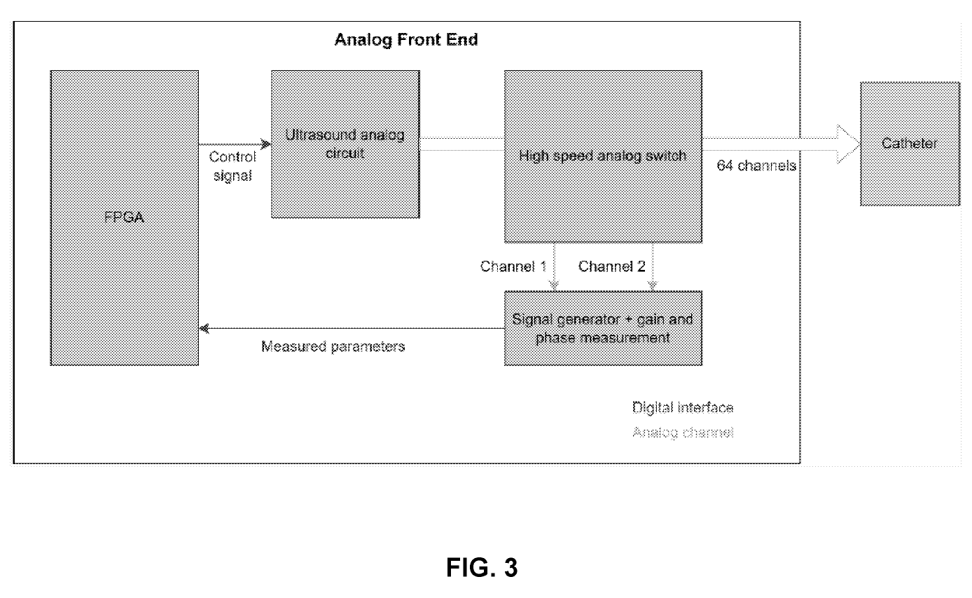

FIG. 3 diagrams an AFE integrated with a catheter ultrasound system, for example, an intravascular ultrasound system. The AFE in this embodiment utilizes a continuous solution according to one embodiment of systems of the invention.

Aspects of the invention include a system comprising an analog front-end (AFE) operably couplable to a multi-element transducer of an ultrasound imaging device via a plurality of element channels. Further, systems of the invention may include a console operably coupled to the AFE and further configured to be operably associated with the ultrasound imaging device and to exchange data therewith The console may include a hardware processor coupled to non-transitory, computer-readable memory containing instructions executable by the processor. The instructions executable by the process may cause the AFE to automatically detect one or more dead element channels when the AFE is coupled to the multi-element transducer of an ultrasound imaging device, and mask the detected one or more dead element channels such that, during an ultrasound pulse cycle, application of an ultrasound pulse to the one or more dead element channels is prevented. Thus, masking the one or more dead element channels allows for pulsing at higher volts.

In some embodiments, masking is performed on receiving. In this way, dead channels, which contribute to darken the image during beamforming are masked.

The smart AFE may be integrated and/or coupled to any ultrasound imaging device. In non-limiting examples, systems of the invention may be coupled to an intravascular ultrasound ultrasonic imaging (IVUS) device. The AFE may be integrated with the IVUS device.

Ultrasound transducers typically consist of 128-512 piezoelectric elements arranged in linear or curvilinear arrays. Each element may be individually insulated. Alternatively, the transducer may produce an ultrasound beam by a mechanical means, via either oscillating or rotating wheels.

The systems of the invention may utilize multiple fields-of-view, e.g. cylindrical fields-of-view acquired with a rotating transducer array or a cylindrical folded transducer matrix. Thus, cylindrical/360-degree imaging may be applied to provide an extended field-of view (FOV). In some embodiments, the systems of the invention may utilize an orbital field-of-view around the catheter, which may be different than the planar field-of-view of conventional ICE catheters with a matrix transducer. 3D ICE catheters with forward and with sideways transducer arrays are suitable as long as the field-of-view is sufficiently large to provide for reconstruction of an anatomical region of interest. For example, catheters facing sideways may be used as these catheters also cover the forward direction to some degree due to the opening angle of the ultrasound beam. Additionally and/or alternatively, a side-looking array transducer may be used to obtain full or partial circumferential 3D image data of the target region.

In some embodiments, the AFE of systems of the invention includes an impedance measurement circuit to detect one or more dead element channels. The impedance measurement circuit may include a signal generator and a gain and phase measurement circuit. The AFE may use a preset low voltage, fixed frequency signal to determine if the channel is within the limits of the designed impedance.

Impedance, represented by the symbol Z and measured in ohms (Ω), represents the overall resistance component of elements or circuits to AC signals. Impedance may refer to the impedance in circuits producing voltage and it may refer to the input impedance in circuits receiving voltage. The value of impedance may be determined by the ratio of voltage to current. The method of calculating impedance may vary depending on the circuit configuration.

As is generally known, impedance includes resistance and reactance in electrical circuits. While resistance indicates electrical resistance in DC circuits, impedance shows the overall resistance in AC circuits. Resistance is a value considering only resistors and does not change with frequency. Reactance includes both capacitive and inductive reactance. Denoted as X and measured in Ohms (Ω), reactance is an expression of the extent to which an electronic component, circuit, or system stores and releases energy as the current and voltage fluctuate with each AC cycle. The impedance phase angle for any component is the phase shift between the voltage across that component and current through that component.

Impedance may be measured and represented on a complex plane, with the real axis representing resistance and the imaginary axis representing reactance. Because impedance is a complex measurement, measurements on different axes, such as real, phase, etc., may be obtained. The real axis may represent the portion of the circuit’s opposition to current flow due to resistive components (e.g. resistors). The frequency of the AC signal applied to the circuit may be plotted on the x-axis when measuring impedance. The imaginary axis may represent the opposition to current flow due to reactive components (e.g. capacitors and inductors). The magnitude of the impedance may be plotted on the y-axis to represent the total opposition to current flow. Complex impedance may be represented on a vector of the plane of the real axis and the imaginary axis, with the magnitude representing the total impedance and its angle (phase shift) indicating a phase difference between voltage and current. The phase shift between the voltage and the current may also be represented on the y-axis, indicating the amount of phase shift caused by the current. In this way, impedance may be determined via measurements on the different axes.

Thus, in some embodiments, the impedance measurement circuit may be configured to analyze an element channel against a preset low voltage, fixed frequency signal to determine whether an impedance measurement of an element channel is within a designated impedance limit. In some embodiments, the impedance measurement circuit measures the impedance of the plurality of element channels before every pulse. In some embodiments, the signal generator, in conjunction with the gain and phase measurement circuit, are configured to measure the impedance of the channel whenever a channel is not being used.

In some embodiments, the AFE may further include a field programmable gate array (FPGA). As is generally known, an FPGA integrated circuit may be utilized for parallel processing capability, i.e. processing data in a parallel fashion whereby operations are performed at the same time as opposed to sequentially. Thus, the FPGA may be suited for high performance computing applications (such as AI), to allow the FPGA to deliver higher performance using lower clock speeds thus consuming lower power. The FPGA may be programmed and re-programmed via a software download to adapt to the needs of the system. The FPGA may be configured using a hardware description language (HDL) such that specific FPGA-based functions, as well as the interconnects between those functions, may be contained in the HDL. The description may be compiled to produce an FPGA configuration file such that built-in FPGA resources (memory arrays, PCI cores and the like) are used, as well creating customized logic circuits (adders, multiplexers and other application-specific functions).

The FPGA may be operable to be reconfigured through an architecture consisting of configurable logic blocks (CLBs), also referred to as programmable logic blocks (PLBs), configurable I/O blocks, and programmable interconnects, i.e. custom connections between logic cells across logic blocks, in non-limiting examples and as is generally known. The CLBs may be in a large grain architecture, in which the CLBs contain logic to create a small state machine. The FPGA may include clock circuitry for driving the clock signals to each logic block, and additional logic resources such as arithmetic logic units (ALUs), memory, and decoders. The FPGA may include digital and analog features, including logic densities, flash memory, embedded processors and digital signal processing (DSP) blocks.

The programmable interconnects may be implemented by switch boxes which contain a number of semiconductor switches. The programmable interconnects provide routing pathways between the FPGA’s logic block, allowing the outputs of a specific unit or an input pad to be linked to any other cell or pad within the circuitry. The programmable interconnects may enable programmable routing to configure the FPGA resources for the implementation of a particular task or application. The interface between the FPGA and other external devices may be enabled via input/output (I/O) blocks (IOBs), i.e. programmable input and output resources configured to match the protocols of external devices to which the FPGA may be connected. Signals entering or leaving the FPGA may do so through device pins and associated IOBs. The CLB may contain one or more memory elements, for example, RAM for creating combinatorial logic functions, i.e. lookup tables (LUTs), single-bit flip-flops, or dense memory arrays. The CLB may contain flip-flops for clocked storage elements, along with multiplexers in order to route the logic within the block and to and from external resources. The FPGA may include dedicated on-chip memory, for example, for data buffering and reuse. The FPGA may use one or more Block memory (i.e. block random access memory, block RAM, or BRAM), static random access memory (SRAM) memory arrays, smaller look-up tables (LUTs) and traditional flip-flop elements to provide the necessary storage.

The FPGA may be of any configuration to offer the required configurability, power consumption, programmable elements, and memory necessary to achieve the functions of the system described herein. In non-limiting examples, the FPGA may be one or more of an anti-fuse FPGA, a static random-access memory (SRAM)-based FPGA, a flash-based FPGA, an EEPROM-based FPGA, a Hybrid FPGA, and System-on-chip FPGA. Anti-fuse FPGAs are not reconfigurable, using a one-time programmable element called an antifuse, which is configured by applying a high voltage to create connections between internal wires. SRAM-based FPGAs, which may be configured at run time, use static random-access memory (SRAM) to store configuration instructions and require external memory to hold the configuration code. Flash-based FPGAs store the configuration in nonvolatile flash memory, which is reprogrammable. EEPROM-based FPGAs utilizes an electronically erasable programmable read-only memory (EEPROM) to store the FPGA’s configuration. Hybrid FPGAs utilize a combination of different programmable elements, such as SRAM- or flash-based logic blocks. System-on-chip (SoC) FPGAs integrate programmable logic with hard processor cores, combining the functionality of both an FPGA and an SoC, on a single silicon chip that combines multiple system processing chips like CPUs, GPUs and RAM into one unit.

Other types of integrated circuits as is generally known in the art may be utilized, for example, microcontroller, microprocessors, and application specific integrated circuits (ASIC).

In some embodiments, the AFE may include an ultrasound analog circuit, and a high speed analog switch. The impedance measurement may be related to the FPGA In some embodiments, the FPGA may mask the open channel. In some embodiments, the AFE may measure other parameters in addition or alternative to impedance. These measured parameter may be, for example, capacitance and current.

In the present invention, the transducer may be any type for transmitting and receiving acoustic waves. For example, the transducer may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices may not require bias for achieving imaging sensitivity.

In some embodiments of the systems of the invention, the transducer may be a micro-electromechanical systems (MEMS)-based capacitive micromachined ultrasonic transducer (CMUT) configured as a two-dimensional (2D) array structure. In non-limiting examples, the 2D array may be a flexible structure. Devices of the invention may include a cylindrical imaging array which consists of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13(10) 3873-3882, incorporated herein by reference.

Additionally, and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers of the invention may be made of electrostrictive materials such as an electrostrictive polymer, or any material that can be activated using bias voltage to achieve imaging sensitivity.

The system may include one or more multiplexers. In some embodiments, the AFE comprises a circuit comprising a multiplexer and a measurement device. The multiplexer may sequentially connect each of the element channels with the measurement device. The multiplexer may or may not be part of the FPGA. As is generally known, the multiplexer may transfer input signals to a microprocessor, which receives and processes the data, transmits it to the output devices, and controls the system as a whole. The multiplexer may be referred to as a data selector, i.e. selecting one of n data inputs and forwarding it to the output. The multiplexers may allow polarity selection and reset and clear input selection.

The measurement device may measure the circuit at one or more points to determine the one or more dead element channels and one or more alive channels. In some embodiments, the measurement device may produce a binary output of an element channel as dead or alive. As disclosed herein, in some embodiments, the measurement device is an impedance analyzer. The impedance measurement may be measured at a known frequency with a known value. For example, if the impedance measurement exceeds a threshold value, the element channel is detected as a dead channel by the system. The threshold value may be an input or programmed threshold value. If the element channel is detected as dead, the measurement device may communicate with the AFE such that AFE prevents application of the ultrasound pulse to the element channel.

In some embodiments, the impedance measurement, using a preset low voltage, fixed frequency signal, may be scanned before every pulse. This allows room for the catheter element to fail dynamically without damaging the AFE channels when pulsing at high voltages.

As illustrated in FIG. 3, the signal generator, along with the gain and phase measurement circuit, may measure the impedance of the channel whenever a channel is not being used. This information may then be relayed to the FPGA, which may mask the channels. The measurement time may directly impact the maximum pulse repetition frequency, and thus may be kept as low as possible.

In addition to detecting dead channels, the measured impedance may serve as an input to set the output-impedance of the pulsar. Thus, matching the output impedance to the channel impedance improves the signal strength.

In some embodiments, the AFE is further configured to model crosstalk using the impedance measurements of two element channels. Using two channels, the cross talk may also be monitored and modeled as a calibration step, which may be used during the reconstruction and rendering of images to compensate for the cross talk. As is generally known, in the conversion of electrical energy to acoustic energy and vice versa performed via a transducer array, cross talk of adjacent elements may have its origin in either electrical (capacitive) cross talk or acoustic propagation cross talk. Generally, electrical cross talk is instantaneous, while acoustic cross talk results in a delayed cross talk signal due to the much slower propagation velocity of acoustic waves versus electromagnetic/electrostatic coupling. Thus, systems of the invention may measure the cross talk, i.e. electrical impedance in the return path, and modeled as a calibration step.

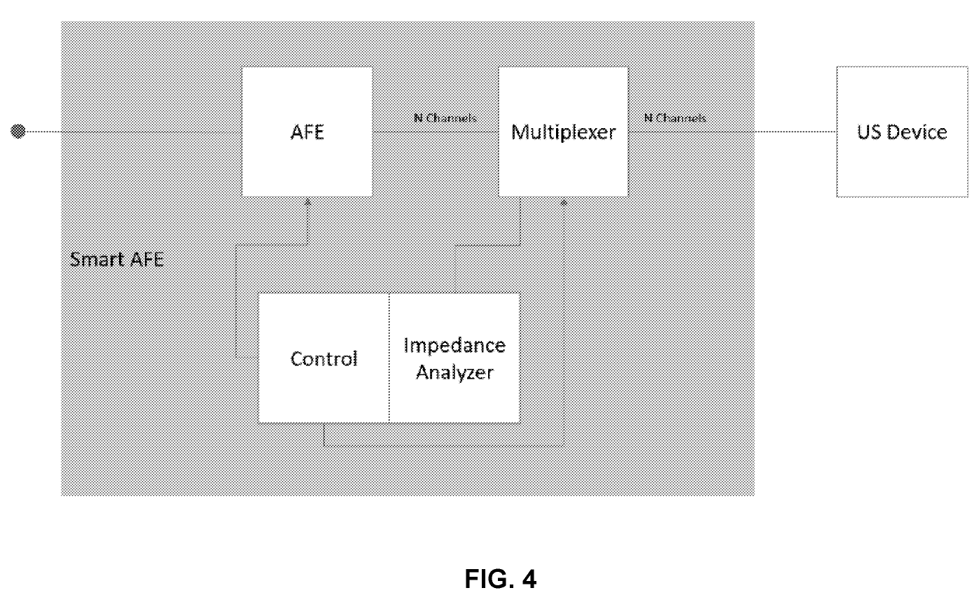

FIG. 4 diagrams a smart AFE according to one embodiment of systems of the invention. The smart AFT may be integrated with a catheter ultrasound system, for example, an intravascular ultrasound system. The AFE in this embodiment utilizes a discrete solution according to one embodiment of systems of the invention.

As disclosed herein, the system may include one or more multiplexers. In some embodiments, the AFE comprises a circuit comprising a multiplexer and a measurement device. The multiplexer may sequentially connect each of the element channels with the measurement device. As is generally known, the multiplexer may transfer input signals to a microprocessor, which receives and processes the data, transmits it to the output devices, and controls the system as a whole. The multiplexer may be referred to as a data selector, i.e. selecting one of n data inputs and forwarding it to the output. The multiplexers may allow polarity selection and reset and clear input selection.

As disclosed herein, the measurement device may be an impedance analyzer or the like. The measurement device may measure the circuit at one or more points to determine the one or more dead element channels and one or more alive channels. In some embodiments, the measurement device may produce a binary output, for example, the binary output may be of an element channel as dead or alive. As disclosed herein, in some embodiments, the measurement device is an impedance analyzer. The impedance measurement may be measured at a known frequency with a known value. For example, if the impedance measurement exceeds a threshold value, the element channel is detected as a dead channel by the system. The threshold value may be an input or programmed threshold value. If the element channel is detected as dead, the measurement device may communicate with the AFE such that AFE prevents application of the ultrasound pulse to the element channel.

In some embodiments, the impedance measurement, using a preset low voltage, fixed frequency signal, may be scanned before every pulse. This allows room for the catheter element to fail dynamically without damaging the AFE channels when pulsing at high voltages.

The measurement device may measure the circuit at certain points, and resolve in a binary output as dead/alive channels. In case of an impedance measurement, the measurement device may measure impedance at a known frequency with a known value. For example, if the impedance value exceeds a threshold value, the channel may be detected as dead. If a channel is detected as dead, the measurement device may notify to the AFE. The AFE will then not pulse anymore on that channel.

### Methods for probe element detection and control using a smart AFE

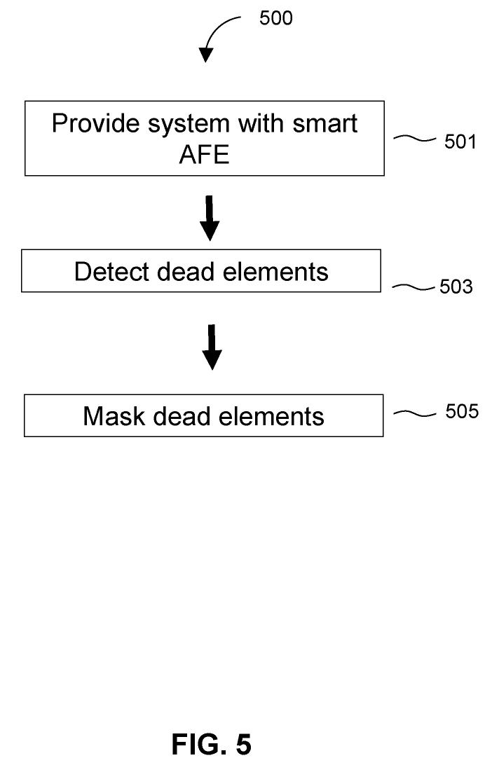

FIG. 5 illustrates a block diagram of one embodiment of methods for probe element detection and control using systems of the invention.

Aspects of the invention include methods for probe element detection and control using systems of the invention. As shown in FIG. 5, in one embodiment, the method 500 includes providing 501 a system comprising an analog front-end (AFE) operably couplable to a multi-element transducer of an ultrasound imaging device via a plurality of element channels and a console operably coupled to the AFE and further configured to be operably associated with the ultrasound imaging device and exchange data therewith; detecting 503 one or more dead element channels when the AFE is coupled to the multi-element transducer of an ultrasound imaging device; and masking 505 the detected one or more dead element channels such that, during an ultrasound pulse cycle, application of an ultrasound pulse to the one or more dead element channels is prevented, wherein masking the one or more dead element channels allows for pulsing at higher volts.

In some embodiments, masking is performed on receiving. In this way, dead channels, which contribute to darken the image during beamforming are masked.

As disclosed herein, the console may include a hardware processor coupled to non-transitory, computer-readable memory containing instructions executable by the processor. The instructions executable by the process may cause the AFE to automatically detect one or more dead element channels when the AFE is coupled to the multi-element transducer of an ultrasound imaging device, and mask the detected one or more dead element channels such that, during an ultrasound pulse cycle, application of an ultrasound pulse to the one or more dead element channels is prevented.

The smart AFE may be integrated and/or coupled to any ultrasound imaging device. In non-limiting examples, systems of the invention may be coupled to an intravascular ultrasound ultrasonic imaging (IVUS) device. The AFE may be integrated with the IVUS device.

Ultrasound transducers typically consist of 128-512 piezoelectric elements arranged in linear or curvilinear arrays. Each element may be individually insulated. Alternatively, the transducer may produce an ultrasound beam by a mechanical means, via either oscillating or rotating wheels.

The methods of the invention may utilize multiple fields-of-view, e.g. cylindrical fields-of-view acquired with a rotating transducer array or a cylindrical folded transducer matrix. Thus, cylindrical/360-degree imaging may be applied to provide an extended field-of view (FOV). In some embodiments, the systems of the invention may utilize an orbital field-of-view around the catheter, which may be different than the planar field-of-view of conventional ICE catheters with a matrix transducer. 3D ICE catheters with forward and with sideways transducer arrays are suitable as long as the field-of-view is sufficiently large to provide for reconstruction of an anatomical region of interest. For example, catheters facing sideways may be used as these catheters also cover the forward direction to some degree due to the opening angle of the ultrasound beam. Additionally and/or alternatively, a side-looking array transducer may be used to obtain full or partial circumferential 3D image data of the target region.

In some embodiments of the methods of the invention, the AFE of systems of the invention may include an impedance measurement circuit to detect one or more dead element channels. The impedance measurement circuit may include a signal generator and a gain and phase measurement circuit. The AFE may use a preset low voltage, fixed frequency signal to determine if the channel is within the limits of the designed impedance.

Impedance, represented by the symbol Z and measured in ohms (Ω), represents the overall resistance component of elements or circuits to AC signals. Impedance may refer to the impedance in circuits producing voltage and it may refer to the input impedance in circuits receiving voltage. The value of impedance may be determined by the ratio of voltage to current. The method of calculating impedance may vary depending on the circuit configuration.

As is generally known, impedance includes resistance and reactance in electrical circuits. While resistance indicates electrical resistance in DC circuits, impedance shows the overall resistance in AC circuits. Resistance is a value considering only resistors and does not change with frequency. Reactance includes both capacitive and inductive reactance. Denoted as X and measured in Ohms (Ω), reactance is an expression of the extent to which an electronic component, circuit, or system stores and releases energy as the current and voltage fluctuate with each AC cycle. The impedance phase angle for any component is the phase shift between the voltage across that component and current through that component.

Thus, in some embodiments of the methods, the impedance measurement circuit may be configured to analyze an element channel against a preset low voltage, fixed frequency signal to determine whether an impedance measurement of an element channel is within a designated impedance limit. In some embodiments, the impedance measurement circuit measures the impedance of the plurality of element channels before every pulse. In some embodiments, the signal generator, in conjunction with the gain and phase measurement circuit, are configured to measure the impedance of the channel whenever a channel is not being used.

Impedance may be measured and represented on a complex plane, with the real axis representing resistance and the imaginary axis representing reactance. Because impedance is a complex measurement, measurements on different axes, such as real, phase, etc., may be obtained. The real axis may represent the portion of the circuit’s opposition to current flow due to resistive components (e.g. resistors). The frequency of the AC signal applied to the circuit may be plotted on the x-axis when measuring impedance. The imaginary axis may represent the opposition to current flow due to reactive components (e.g. capacitors and inductors). The magnitude of the impedance may be plotted on the y-axis to represent the total opposition to current flow. Complex impedance may be represented on a vector of the plane of the real axis and the imaginary axis, with the magnitude representing the total impedance and its angle (phase shift) indicating a phase difference between voltage and current. The phase shift between the voltage and the current may also be represented on the y-axis, indicating the amount of phase shift caused by the current. In this way, impedance may be determined via measurements on the different axes.

In some embodiments of the methods of the invention, the AFE may further include a field programmable gate array (FPGA). As is generally known, an FPGA integrated circuit may be utilized for parallel processing capability, i.e. processing data in a parallel fashion whereby operations are performed at the same time as opposed to sequentially. Thus, the FPGA may be suited for high performance computing applications (such as AI), to allow the FPGA to deliver higher performance using lower clock speeds thus consuming lower power. The FPGA may be programmed and re-programmed via a software download to adapt to the needs of the system. The FPGA may be configured using a hardware description language (HDL) such that specific FPGA-based functions, as well as the interconnects between those functions, may be contained in the HDL. The description may be compiled to produce an FPGA configuration file such that built-in FPGA resources (memory arrays, PCI cores and the like) are used, as well creating customized logic circuits (adders, multiplexers and other application-specific functions).

The FPGA may be operable to be reconfigured through an architecture consisting of configurable logic blocks (CLBs), also referred to as programmable logic blocks (PLBs), configurable I/O blocks, and programmable interconnects, i.e. custom connections between logic cells across logic blocks, in non-limiting examples and as is generally known. The CLBs may be in a large grain architecture, in which the CLBs contain logic to create a small state machine. The FPGA may include clock circuitry for driving the clock signals to each logic block, and additional logic resources such as arithmetic logic units (ALUs), memory, and decoders. The FPGA may include digital and analog features, including logic densities, flash memory, embedded processors and digital signal processing (DSP) blocks.

The programmable interconnects may be implemented by switch boxes which contain a number of semiconductor switches. The programmable interconnects provide routing pathways between the FPGA’s logic block, allowing the outputs of a specific unit or an input pad to be linked to any other cell or pad within the circuitry. The programmable interconnects may enable programmable routing to configure the FPGA resources for the implementation of a particular task or application. The interface between the FPGA and other external devices may be enabled via input/output (I/O) blocks (IOBs), i.e. programmable input and output resources configured to match the protocols of external devices to which the FPGA may be connected. Signals entering or leaving the FPGA may do so through device pins and associated IOBs. The CLB may contain one or more memory elements, for example, RAM for creating combinatorial logic functions, i.e. lookup tables (LUTs), single-bit flip-flops, or dense memory arrays. The CLB may contain flip-flops for clocked storage elements, along with multiplexers in order to route the logic within the block and to and from external resources. The FPGA may include dedicated on-chip memory, for example, for data buffering and reuse. The FPGA may use one or more Block memory (i.e. block random access memory, block RAM, or BRAM), static random access memory (SRAM) memory arrays, smaller look-up tables (LUTs) and traditional flip-flop elements to provide the necessary storage.

The FPGA may be of any configuration to offer the required configurability, power consumption, programmable elements, and memory necessary to achieve the functions of the system described herein. In non-limiting examples, the FPGA may be one or more of an anti-fuse FPGA, a static random-access memory (SRAM)-based FPGA, a flash-based FPGA, an EEPROM-based FPGA, a Hybrid FPGA, and System-on-chip FPGA. Anti-fuse FPGAs are not reconfigurable, using a one-time programmable element called an antifuse, which is configured by applying a high voltage to create connections between internal wires. SRAM-based FPGAs, which may be configured at run time, use static random-access memory (SRAM) to store configuration instructions and require external memory to hold the configuration code. Flash-based FPGAs store the configuration in nonvolatile flash memory, which is reprogrammable. EEPROM-based FPGAs utilizes an electronically erasable programmable read-only memory (EEPROM) to store the FPGA’s configuration. Hybrid FPGAs utilize a combination of different programmable elements, such as SRAM- or flash-based logic blocks. System-on-chip (SoC) FPGAs integrate programmable logic with hard processor cores, combining the functionality of both an FPGA and an SoC, on a single silicon chip that combines multiple system processing chips like CPUs, GPUs and RAM into one unit.

Other types of integrated circuits as is generally known in the art may be utilized, for example, microcontroller, microprocessors, and application specific integrated circuits (ASIC).

In some embodiments of the methods, the AFE may include an ultrasound analog circuit, and a high speed analog switch. The impedance measurement may be related to the FPGA In some embodiments, the FPGA may mask the open channel. In some embodiments, the AFE may measure other parameters in addition or alternative to impedance. These measured parameter may be, for example, capacitance and current.

In the present invention, the transducer may be any type for transmitting and receiving acoustic waves. For example, the transducer may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices may not require bias for achieving imaging sensitivity.

In some embodiments of the methods of the invention, the transducer may be a micro-electromechanical systems (MEMS)-based capacitive micromachined ultrasonic transducer (CMUT) configured as a two-dimensional (2D) array structure. In non-limiting examples, the 2D array may be a flexible structure. Devices of the invention may include a cylindrical imaging array which consists of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13(10) 3873-3882, incorporated herein by reference.

Additionally, and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers of the invention may be made of electrostrictive materials such as an electrostrictive polymer, or any material that can be activated using bias voltage to achieve imaging sensitivity.

The system may include one or more multiplexers. In some embodiments of the methods, the AFE comprises a circuit comprising a multiplexer and a measurement device. The multiplexer may sequentially connect each of the element channels with the measurement device. The multiplexer may or may not be part of the FPGA. As is generally known, the multiplexer may transfer input signals to a microprocessor, which receives and processes the data, transmits it to the output devices, and controls the system as a whole. The multiplexer may be referred to as a data selector, i.e. selecting one of n data inputs and forwarding it to the output. The multiplexers may allow polarity selection and reset and clear input selection.

The measurement device may measure the circuit at one or more points to determine the one or more dead element channels and one or more alive channels. In some embodiments, the measurement device may produce a binary output of an element channel as dead or alive. As disclosed herein, in some embodiments, the measurement device is an impedance analyzer. The impedance measurement may be measured at a known frequency with a known value. For example, if the impedance measurement exceeds a threshold value, the element channel is detected as a dead channel by the system. The threshold value may be an input or programmed threshold value. If the element channel is detected as dead, the measurement device may communicate with the AFE such that AFE prevents application of the ultrasound pulse to the element channel.

In some embodiments of the methods, the impedance measurement, using a preset low voltage, fixed frequency signal, may be scanned before every pulse. This allows room for the catheter element to fail dynamically without damaging the AFE channels when pulsing at high voltages.

Referring again to FIG. 3, the signal generator, along with the gain and phase measurement circuit, may measure the impedance of the channel whenever a channel is not being used. This information may then be relayed to the FPGA, which may mask the channels. The measurement time may directly impact the maximum pulse repetition frequency, and thus may be kept as low as possible.

In addition to detecting dead channels, the measured impedance may serve as an input to set the output-impedance of the pulsar. Thus, matching the output impedance to the channel impedance improves the signal strength.

In some embodiments of the methods, the AFE is further configured to model crosstalk using the impedance measurements of two element channels. Using two channels, the cross talk may also be monitored and modeled as a calibration step, which may be used during the reconstruction and rendering of images to compensate for the cross talk. As is generally known, in the conversion of electrical energy to acoustic energy and vice versa performed via a transducer array, cross talk of adjacent elements may have its origin in either electrical (capacitive) cross talk or acoustic propagation cross talk. Generally, electrical cross talk is instantaneous, while acoustic cross talk results in a delayed cross talk signal due to the much slower propagation velocity of acoustic waves versus electromagnetic/electrostatic coupling. Thus, systems of the invention may measure the cross talk, i.e. electrical impedance in the return path, and modeled as a calibration step.

Referring again to FIG. 4, which diagrams a smart AFE according to one embodiment of systems of the invention, the system may include one or more multiplexors. The smart AFT may be integrated with a catheter ultrasound system, for example, an intravascular ultrasound system. The AFE in this embodiment utilizes a discrete solution according to one embodiment of systems of the invention.

In some embodiments, the AFE comprises a circuit comprising a multiplexer and a measurement device. The multiplexer may sequentially connect each of the element channels with the measurement device. As is generally known, the multiplexer may transfer input signals to a microprocessor, which receives and processes the data, transmits it to the output devices, and controls the system as a whole. The multiplexer may be referred to as a data selector, i.e. selecting one of n data inputs and forwarding it to the output. The multiplexers may allow polarity selection and reset and clear input selection.

As disclosed herein, the measurement device may be an impedance analyzer or the like. The measurement device may measure the circuit at one or more points to determine the one or more dead element channels and one or more alive channels. In some embodiments, the measurement device may produce a binary output, for example, the binary output may be of an element channel as dead or alive. As disclosed herein, in some embodiments, the measurement device is an impedance analyzer. The impedance measurement may be measured at a known frequency with a known value. For example, if the impedance measurement exceeds a threshold value, the element channel is detected as a dead channel by the system. The threshold value may be an input or programmed threshold value. If the element channel is detected as dead, the measurement device may communicate with the AFE such that AFE prevents application of the ultrasound pulse to the element channel.

In some embodiments of the methods, the impedance measurement, using a preset low voltage, fixed frequency signal, may be scanned before every pulse. This allows room for the catheter element to fail dynamically without damaging the AFE channels when pulsing at high voltages.

The measurement device may measure the circuit at certain points, and resolve in a binary output as dead/alive channels. In case of an impedance measurement, the measurement device may measure impedance at a known frequency with a known value. For example, if the impedance value exceeds a threshold value, the channel may be detected as dead. If a channel is detected as dead, the measurement device may notify to the AFE. The AFE will then not pulse anymore on that channel.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions, or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term "non-transitory" is to be understood to remove only propagating transitory signals per se from the claim scope and does not relinquish rights to all standard computer-readable media that are not only propagating transitory signals per se. Stated another way, the meaning of the term "non-transitory computer-readable medium" and "non-transitory computer-readable storage medium" should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system comprising:
an analog front-end (AFE) operably couplable to a multi-element transducer of an ultrasound imaging device via a plurality of element channels; and
a console operably coupled to the AFE and further configured to be operably associated with the ultrasound imaging device and exchange data therewith, wherein the console comprises a hardware processor coupled to non-transitory, computer-readable memory containing instructions executable by the processor to cause the AFE to:
automatically detect one or more dead element channels when the AFE is coupled to the multi-element transducer of an ultrasound imaging device; and
mask the detected one or more dead element channels such that, during an ultrasound pulse cycle, application of an ultrasound pulse to the one or more dead element channels is prevented, wherein masking the one or more dead element channels allows for pulsing at higher volts.

2. The system of claim 1, wherein the AFE comprises an impedance measurement circuit comprising a signal generator and gain and phase measurement circuit, wherein the impedance measurement circuit detects the one or more dead element channels.

3. The system of claim 2, wherein the impedance measurement circuit is configured to analyze an element channel against a preset low voltage, fixed frequency signal to determine whether an impedance measurement of an element channel is within a designated impedance limit.

4. The system of claim 3, wherein the impedance measurement circuit measures the impedance of the plurality of element channels before every pulse.

5. The system of claim 3, wherein the signal generator, in conjunction with the gain and phase measurement circuit, are configured to measure the impedance of the channel whenever a channel is not being used.

6. The system of claim 3, wherein the AFE further comprises a field programmable gate array (FPGA), an ultrasound analog circuit, and a high speed analog switch, wherein the impedance measurement is relayed to the FPGA, wherein the FPGA masks the open channel.

7. The system of claim 3, wherein the impedance measurement is used as an input to set the output impedance of an ultrasound pulser, wherein matching the output impedance to an element channel impedance improves signal strength.

8. The system of claim 3, wherein the AFE is further configured model a crosstalk using the impedance measurements of two element channels.

9. The system of claim 8, wherein the modeled crosstalk is used for calibration in one or more of image reconstruction and/or rendering.

10. The system of claim 1, wherein the AFE comprises a circuit comprising a multiplexer and a measurement device, wherein the multiplexer sequentially connects each of the element channels with the measurement device.

11. The system of claim 10, wherein the measurement device measures the circuit at one or more points to determine the one or more dead element channels and one or more alive channels, wherein the measurement device produces a binary output of an element channel as dead or alive.

12. The system of claim 10, wherein the measurement device is an impedance analyzer.

13. The system of claim 12, wherein the impedance measurement is measured at a known frequency with a known value.

14. The system of claim 13, wherein the element channel is detected as a dead channel if the impedance measurement exceeds a threshold value.

15. The system of claim 14, wherein when an element channel is detected as dead, the measurement device communicates with the AFE such that AFE prevents application of the ultrasound pulse to the element channel.

16. A method comprising:
providing a system comprising an analog front-end (AFE) operably couplable to a multi-element transducer of an ultrasound imaging device via a plurality of element channels and a console operably coupled to the AFE and further configured to be operably associated with the ultrasound imaging device and exchange data therewith;
detecting one or more dead element channels when the AFE is coupled to the multi-element transducer of an ultrasound imaging device; and
masking the detected one or more dead element channels such that, during an ultrasound pulse cycle, application of an ultrasound pulse to the one or more dead element channels is prevented, wherein masking the one or more dead element channels allows for pulsing at higher volts.

17. The method of claim 16, wherein the AFE comprises an impedance measurement circuit comprising a signal generator and gain and phase measurement circuit, wherein the impedance measurement circuit detects the one or more dead element channels.

18. The method of claim 17, wherein the impedance measurement circuit is configured to analyze an element channel against a preset low voltage, fixed frequency signal to determine whether an impedance measurement of an element channel is within a designated impedance limit.

19. The method of claim 18, wherein the impedance measurement circuit measures the impedance of the plurality of element channels before every pulse.

20. The method of claim 18, wherein the signal generator, in conjunction with the gain and phase measurement circuit, are configured to measure the impedance of the channel whenever a channel is not being used.

21. The method of claim 18, wherein the AFE further comprises a field programmable gate array (FPGA), an ultrasound analog circuit, and a high speed analog switch, wherein the impedance measurement is relayed to the FPGA, wherein the FPGA masks the open channel.

22. The method of claim 18, wherein the impedance measurement is used as an input to set the output impedance of an ultrasound pulser, wherein matching the output impedance to an element channel impedance improves signal strength.

23. The method of claim 18, wherein the AFE is further configured model a crosstalk using the impedance measurements of two element channels.

24. The method of claim 23, wherein the modeled crosstalk is used for calibration in one or more of image reconstruction and/or rendering.

25. The method of claim 16, wherein the AFE comprises a circuit comprising a multiplexer and a measurement device, wherein the multiplexer sequentially connects each of the element channels with the measurement device.

26. The method of claim 25, wherein the measurement device measures the circuit at one or more points to determine the one or more dead element channels and one or more alive channels, wherein the measurement device produces a binary output of an element channel as dead or alive.

27. The method of claim 25, wherein the measurement device is an impedance analyzer.

28. The method of claim 27, wherein the impedance measurement is measured at a known frequency with a known value.

29. The method of claim 28, wherein the element channel is detected as a dead channel if the impedance measurement exceeds a threshold value.

30. The method of claim 29, wherein when an element channel is detected as dead, the measurement device communicates with the AFE such that AFE prevents application of the ultrasound pulse to the element channel.
